(19) United States
(12) Patent Application Publication
(10) Pub. No.: US 2008/0152613 A1
(43) Pub. Date: Jun. 26, 2008

(54) METHOD FOR MANUFACTURING A SKIN CARE COMPOSITION

(76) Inventor: Robert B. Norburn, Hammonds Plains (CA)

Correspondence Address: MARK R. MALEK, ESQUIRE;THE TORPY GROUP, P.L., SUITE 200, 202 N. HARBOR CITY BLVD., MELBOURNE, FL, 32935, US

(21) Appl. No.: 11/931,871

(22) Filed: Oct. 31, 2007

Related U.S. Application Data
(63) Continuation-in-part of application No. 10/349,116, filed on Jan. 23, 2003, now Pat. No. 7,354,573.
(60) Provisional application No. 60/352,571, filed on Jan. 31, 2002.

(51) Int. Cl. A61K 8/18; A61K 35/64; A61Q 9/02

(52) U.S. Cl. 424/73; 424/539

(57) ABSTRACT

A method for manufacturing a skin care composition may include blending a first phase of the skin care composition. The first phase of the skin care composition may include a predetermined percentage of water, between about 2% and 20% by weight glycerin and/or diglycerin, and between about 0.1% and 5% by weight of an emulsifier. The method may also include heating the first phase to a first predetermined temperature. The method may further include blending a second phase of the skin care composition. The second phase of the skin care composition may include between about 2% to 20% by weight grapeseed oil, between about 2% to 30% by weight stearic acid, between about 0.5% and 5% by weight shea butter, between about 0.5% to 5% by weight beeswax and/or synthetic beeswax, between about 0.5% to 2% by weight cetyl alcohol, and between about 0.5% to 5% by weight essential oil. The method may still further include heating the second phase to a second predetermined temperature, blending the first phase at the first predetermined temperature to the second phase at second predetermined temperature, and cooling the blended first and second phase to a third predetermined temperature. All weights are based on a total weight of the composition.

### RELATED APPLICATIONS

This application is a continuation-in-part of U.S. patent application Ser. No. 10/349,116 filed on Jan. 23, 2003 which claims the benefit of U.S. Provisional Patent Application No. 60/352,571 filed on Jan. 31, 2002, and is related to co-pending U.S. patent application Ser. No. ______ titled “Skin Care Composition” and filed concurrently herewith, the contents of each of which are incorporated herein in their entireties.

### FIELD OF THE INVENTION

The present invention is related to skin care products, particularly to shaving, after-shave and skin conditioning compositions.

### BACKGROUND OF THE INVENTION

Except for the introduction of electrical or mechanical devices, shaving methods have changed little over the centuries, the traditional wet shave still being predominant. Attempts are made to soften the hair by the application of wetted soap lathers, foams, gels or oils of various natures to make the hair somewhat easier to cut. These pre-shaving applications do little if anything to alleviate the problems encountered when shaving, specifically, razor bumps (pseudo folliculitis barbae), razor burn (microscopic cuts that easily become infected or inflamed), nicking or more serious cutting of the skin, and premature wear on the cutting edge of the blade being used. The applications mentioned above (soap lathers, gels, etc) usually require the use of additional ‘after-shave’ products of one form or another to calm or condition the skin after shaving.

U.S. Pat. No. 4,608,392 describes the use of polyether oligomers in a skin care product.

U.S. Pat. No. 6,264,963 describes a skin care composition whose benefits are achieved by using petrolatum or mineral oil. Although this patent mentions “softening of the hair prior to cutting” it makes no claim or suggestion that their formula could be used for shaving.

U.S. Pat. No. 4,784,849 and corresponding Canadian Patent 1,244,350 describe skin care and shaving compositions comprising phospholipids.

U.S. Pat. No. 5,345,680 describes a shaving aid for wet razor comprising polymers.

U.S. Pat. No. 5,902,574 describes a shaving preparation that uses cysteamine.

British Patent 429,786 describes a shaving preparation comprising sulphonated oils.

British Patent 454,658 describes a shaving cream, however, there is no mention of the use of either glycerin or diglycerin.

### BRIEF SUMMARY OF THE INVENTION

in view of the foregoing background, it is therefore an object of the present invention to provide a method for manufacturing a skin care composition that provides high moisturizing properties, healing properties and lubrication properties. Accordingly, the skin care composition manufactured by the method of the present invention may advantageously have a number of uses such as, for example, shaving preparation, shaving, aftershave, skin moisturizer, and skin healing.

The method for manufacturing the skin care composition may include blending a first phase of the skin care composition. The first phase may include a predetermined percentage of water, between about 2% and 20% by weight glycerin and/or diglycerin, and between about 0.1% and 5% by weight of an emulsifier. The method may also include heating the first phase to a first predetermined temperature.

The method may further include blending a second phase of the skin care composition. The second phase may include between about 2% to 20% by weight grapeseed oil, between about 2% to 30% by weight stearic acid, between about 0.5% and 5% by weight shea butter, between about 0.5% to 5% by weight beeswax and/or synthetic beeswax, between about 0.5% to 2% by weight cetyl alcohol, and between about 0.5% to 5% by weight essential oil.

The method may still further include heating the second phase to a second predetermined temperature. The method may also include blending the first phase at the first predetermined temperature to the second phase at second predetermined temperature, and cooling the blended first and second phase to a third predetermined temperature. All weights are based on a total weight of the composition.

The first phase and the second phase may be combined using shear mixing. Further, the first phase and second phase may be blended together until a uniform cream is formed. The method may also include adding between about 0.5% to 5% by weight of a pH balancer to the skin care composition. More particularly, the pH balancer may be added to either the first phase of the skin care composition or to the blended first and second phase of the skin care composition.

The method may also further comprise stirring the blended first and second phase while cooling to the third predetermined temperature, and blending a predetermined percentage by weight of preservatives and/or additives with the blended first and second phase. The preservatives and additives may be blended with the first and second phase using shear mixing, and the additives may be selected from the group consisting of a perfume, a coloring agent, a UV blocker, a skin protectant, an insect repellent, a medication, a fragranced oil, or any combination thereof. The fragranced oil may, for example, be orange oil.

The emulsifier may be one selected from the group consisting of glyceryl stearate and a carbomer, and the preservative may be grapefruit seed extract that is present in an amount of 0.1% to 2% by weight. The stearic acid may be present in an amount of 10% to 15% by weight.

The first predetermined temperature may be between about 65 degrees Celsius and 75 degrees Celsius. The second predetermined temperature may be between about 70 degrees Celsius and 80 degrees Celsius, and the third predetermined temperature may be between about 40 degrees Celsius and 50 degrees Celsius.

### DETAILED DESCRIPTION OF THE INVENTION

The amount of water (preferably water which has been deionised, distilled or otherwise purified) utilized in the composition may vary dependent on the desired consistency/viscosity of the final product. Water is generally present in an amount of about 20% to about 85% by weight, more particularly from about 50% to about 75% by weight and even more particularly from about 55% to about 70% by weight of the total composition.

Stearic acid is a commercial product and may be obtained from a variety of sources well known to those skilled in the art. It is generally present in an amount of about 2% to about 40% by weight, more particularly from about 5% to about 35% by weight and even more particularly from about 15% to about 30% by weight of the total composition.

Glycerin and diglycerin are also commercial products and may be obtained from a variety of sources well known to those skilled in the art. When present in the composition, they are generally present in an amount of about 2% to about 40% by weight, more particularly from about 5% to about 25% by weight and even more particularly from about 10% to about 20% by weight of the total composition.

Grapeseed oil is also a commercial product and may be obtained from a variety of sources well known to those skilled in the art. When present in the composition, it is generally present in an amount of about 2% to about 40% by weight, particularly about 2% to about 20% by weight, more particularly from about 5% to about 15% by weight and even more particularly from about 10% to about 15% by weight of the total composition. Grapeseed oil is an anti-oxidant and may also help to lubricate during shaving. It is also a known and documented anti-carcinogen.

One of glycerin, diglycerin and grapeseed oil may be present individually in the composition, or a combination of two or more of glycerin, diglycerin and grapeseed oil may be present. It is possible to make glycerin and diglycerin from grapeseed oil by known processes. Any glycerin or diglycerin used in the composition may be made partially or entirely from grapeseed oil. This is especially useful when grapeseed oil itself is not used in the composition.

The composition may contain small amounts of other ingredients (generally 5% or less of total weight, although greater amounts are possible in some instances). Other ingredients include, but are not limited to, for example emulsifiers (e.g. glyceryl stearate), preservatives (e.g. grapefruit seed extract (GSE)), perfumes (e.g. Bergamot oil), colouring agents, UV blockers, skin protectants (e.g. zinc oxide, titanium oxide, and the like), insect repellents, medications, pH modifiers (e.g. sodium hydroxide and/or triethanolamine), or a combination thereof. Typically, other ingredients may be present in an amount of less than 2% by weight, although, for example, an emulsifier may be present in an amount as much as 10% by weight and a preservative, for example GSE, may be present in an amount of about 0.1% to about 5% by weight, with an amount of about 0.1% to about 2% by weight GSE being normal for use as a preservative in many applications. These ingredients are well known in the field of skin care and the proportions are established and known to those skilled in the art.

The composition may also include a carbomer which, as is known by those skilled in the art, may be used as an emulsion stabilizer or thickening agent in some cosmetics.

Preservatives may act as product stabilizers and/or as shelf life enhancers and/or as anti-microbials. While any suitable preservative may be used, grapefruit seed extract (GSE) is preferred since it is a natural organic ingredient rather than of synthetic origin. Grapefruit seed extract (GSE) is well known and widely used for its anti-bacterial, anti-fungal, anti-viral and disinfectant properties. Those skilled in the art will appreciate that any food grade preservative may be used in the composition. Several preservatives are well known for use in cosmetics and are preferable for use in the present composition. One example of a preservative is provided under the tradename Geogard™ and manufactured by Lonza™.

The composition may also contain bees wax and/or synthetic bees wax in a small percentage. More specifically, the bees wax and/or synthetic bees wax may be present in an amount of between about 0.1% and 10% by weight of the total composition. Other additives may include shea butter, which may be present in an amount of between about 0.1% to 10% by weight of the total composition, and cetyl alcohol, which may be present in an amount of between about 0.1% to 5% by weight of the total composition.

Other additives may be present in the composition and may include essential oils or fragranced oil. The essential oils and/or fragranced oils may include, for example, orange oil, which may be present in the composition in an amount between about 0.1% 10% by weight of the total composition, and soybean oil, which may be present in the composition in an amount between about 0.1% 10% by weight of the total composition.

Triethanolamine may also be present in composition of the present invention. Those skilled in the art will appreciate that triethanolamine is used as a pH balancer in cosmetic preparations.

The composition of the present invention is easily formulated by methods known in the art. Generally, the ingredients may be blended in the desired proportions to form the composition and then packaged into a suitable container, together with instructions for its use. Furthermore, the composition is easily packaged as a non-pressurized product, safe for air travel.

In a particular embodiment, the composition of the present invention may be prepared in a stage-wise blending procedure. For example, a first phase containing stearic acid, and optionally grapeseed oil, glyceryl stearate, and/or cetyl alcohol is heated and blended with a propeller mixer in a jacketed vessel, at a temperature that permits homogenisation, typically from about 60 degrees Celsius to about 80 degrees Celsius more particularly 70 degrees Celsius. A second phase containing water, glycerin and/or diglycerin, carbomer and optionally other ingredients such as grapefruit seed extract, is heated and blended with a propeller mixer in a separate jacketed vessel, at a mixing temperature that permits easy homogenisation, typically from about 60 degrees Celsius to about 80 degrees Celsius, more particularly about 70 degrees Celsius. Once both phases are homogeneous, the first phase may be added to the second phase by blending, for example with a Silverson™ type mixer, for a period of time to permit substantially thorough blending (e.g. for about 1 hour depending on batch size). If desired, a pH modifier may be added to the mixture of the first phase, second phase, or third phase to adjust the pH to 6.0-6.5, and the mixture blended with a propeller mixer before blending in the Silverson™ type mixer. After mixing with the Silverson™ type mixer, the product may then be returned to a propeller mixer and blended until the product has cooled sufficiently to permit the addition of any other desired ingredients. The third phase containing any other desired ingredients may then be thoroughly dispersed in the product by introduction and blending as appropriate, and the product then left to cool.

The present invention advantageously includes methods for manufacturing the skin care composition. The method may include blending a first phase of the skin care composition. The first phase of the skin care composition may include a predetermined percentage of water, between about 2% and 20% by weight glycerin and/or diglycerin, and between about 0.1% and 5% by weight of an emulsifier. The method may also include heating the first phase to a first predetermined temperature.

The method may further include blending a second phase of the skin care composition. The second phase of the skin care composition may include between about 2% to 20% by weight grapeseed oil, between about 2% to 30% by weight stearic acid, between about 0.5% and 5% by weight shea butter, between about 0.5% to 5% by weight beeswax and/or synthetic beeswax, between about 0.5% to 2% by weight cetyl alcohol, and between about 0.5% to 5% by weight essential oil. The method may still further include heating the second phase to a second predetermined temperature.

The method may also include adding between about 0.5% to 2.0% of a pH balancer to any phase of the skin care composition. More particularly, the pH balancer may, for example, be added to the first phase of the skin care composition, or may be added to the blended first and second phase of the skin care composition. The method may stilt further include blending the first phase at the first predetermined temperature to the second phase at second predetermined temperature. The method may further include cooling the blended first and second phase to a third predetermined temperature. All weights are based on a total weight of the composition.

The first phase and the second phase may be combined using shear mixing. Further, the first phase and second phase may be blended together until a uniform cream is formed.

The method may also further comprise stirring the blended first and second phase while cooling to the third predetermined temperature, and blending a predetermined percentage by weight of preservatives and/or additives with the blended first and second phase. The preservatives and additives may be blended with the first and/or second phase using shear mixing, and the additives may be selected from the group consisting of a perfume, a coloring agent, a UV blocker, a skin protectant, an insect repellent, a medication, a fragranced oil, or any combination thereof. The fragranced oil may, for example, be orange oil.

The emulsifier may be one selected from the group consisting of glyceryl stearate and a carbomer, and the preservative may be grapefruit seed extract that is present in an amount of 0.1% to 2% by weight. The stearic acid may be present in an amount of 10% to 15% by weight.

The first predetermined temperature may be between about 65 degrees Celsius and 75 degrees Celsius. The second predetermined temperature may be between about 70 degrees Celsius and 80 degrees Celsius, and the third predetermined temperature may be between about 40 degrees Celsius and 50 degrees Celsius.

Another method for manufacturing a skin care composition may comprise blending a first phase of the skin care composition. The first phase of the skin care composition may include a predetermined percentage of water, and between about 2% and 20% by weight glycerin and/or diglycerin. The method may also include heating the first phase to a first predetermined temperature.

The method may further include blending a second phase of the skin care composition. The second phase of the skin care composition may include between about 2% to 20% by weight grapeseed oil and/or soybean oil, between about 2% to 30% by weight stearic acid, between about 0.5% and 5% by weight shea butter, between about 0.5% to 5% by weight beeswax and/or synthetic beeswax, and between about 0.5% to 2% by weight cetyl alcohol. The method may still further include heating the second phase to a second predetermined temperature and blending the first phase at the first predetermined temperature to the second phase at second predetermined temperature. The method may also include cooling the blended first and second phase to a third predetermined temperature. All weights are based on a total weight of the composition.

Those skilled in the art will appreciate that the first and second phases of the skin care composition may include several variations as disclosed, for example, throughout this specification.

The composition of the present invention preferably is used as a shaving lubricant, as an after-shave and/or as a skin conditioner that moisturizes, conditions and/or protects the skin, leaving an invisible barrier that provides a pleasant and non-greasy tactile experience. The composition is particularly useful as a multi-purpose cream that not only provides a very comfortable and close shaving experience, but also acts as an after-shave, conditioning and protecting the skin. More particularly, the composition is useful as a shaving cream.

Advantageously, the composition may provide a high degree of lubricity for shaving, plus emollient and humectant properties when used to condition the skin. The compositions are easily applied and quickly absorbed by both the hair and the skin. The composition reduces the need for additional after-shave compositions, in particular, reducing or eliminating the need or reliance on alcohol used in many after-shave products.

The composition of the present invention softens the hair and lubricates the skin, reducing the drag or friction of the blade against the hair or the skin, making it much easier for a blade to cut the hair. The high lubricity provided by the composition reduces the incidence of razor bumps, razor burns nicks and cutting of the skin whilst at the same time protecting the cutting edge of the blade from excessive wear thus extending the effective life of the razor. The composition being readily absorbed by the skin is less likely to be dragged off during repeated shaving strokes, as is the case when using soap lathers, foams or other similar products, therefore providing a better and less problematic shave. The composition reduces or eliminates the need for soaps, foams, gels and other pre-shave lubricants and also reduces or eliminates the need to use water when shaving.

Being a non-foaming application, the composition also allows a person shaving to see more readily where the blade is cutting, which is particularly appreciated by those who have a partial beard or moustache.

However, since the composition is an excellent skin conditioner, it may be used solely as such, if desired. In particular, the composition provides excellent skin hydration plus the additional benefit of reduction of TEWL (trans epidermal water loss).

To reduce the possibility of drying of the skin, irritation of the skin, dermatitis or any other adverse reactions associated with petroleum or mineral oils these ingredients, which are otherwise commonly utilized in the cosmetic industry, have been intentionally excluded from the skin care composition of the present invention.

The composition of the present invention may also advantageously be used to enhance skin healing. To be specific, the composition of the present invention may advantageously be used to heal skin that has recently been tattooed. A tattoo applied to the skin causes scarring of the skin and application of the composition according to the present invention may advantageously enhance the healing of the tattoo, as well as reduce the risk of infection.

Animal products and/or by-products are also intentionally excluded from the skin care composition of the present invention not simply to avoid known adverse skin reactions but also to avoid any (even theoretical) prospect of contracting a prion disease (e.g., BSE aka Mad Cow Disease) that could be present in animal products or by-products. Furthermore animal products and/or by-products have been intentionally excluded to respect people's religious beliefs and lifestyle choices. The compositions are intended to be both Kosher and Halal. Peanut oil is intentionally avoided so as to eliminate the risk of severe or even fatal allergic reactions. The use of plant based ingredients responds to the demand from the general public for more natural and/or organic products, versus chemical or synthetic concoctions.

Preferably, the composition is intentionally uncomplicated, the intent being to produce a less complex product, comprising a minimal number of ingredients. When compared to other skin care preparations, the composition of the present invention can dramatically reduce the number and amount of chemicals being applied to the skin. Furthermore, the nature of each ingredient is selected so that reasonably educated members of the general public could easily recognize, relate to, and feel comfortable with the selected ingredients. Being a less complex composition, people will more likely read the list of ingredients and be less intimidated by the basic nature of the contents. In addition, the composition has potential cost, convenience and ecological advantages of being a single-pack, non pressurized, multipurpose product.

Thus, in particularly preferred embodiments, the composition consists essentially of water, stearic acid, and glycerin, diglycerin, grapeseed oil or a combination thereof.

In one preferred embodiment, the composition consists essentially of 2-15% stearic acid, 5-20% glycerin and/or 5-10% grapeseed oil, 2-10% glyceryl stearate, 0.5-5% grapefruit seed extract, sodium hydroxide to adjust pH to 6.0-6.5, and water (e.g. deionized) to 100% (i.e. the balance).

In another preferred embodiment, the composition consists essentially of 2-5% stearic acid, 5-10% glycerin, 5-10% grapeseed oil, 2-6% glyceryl stearate, 0.5-1% grapefruit seed extract, sufficient sodium hydroxide (<0.1%) to achieve a pH of 6.0-6.5, and water (e.g. deionized) to 100% (i.e. the balance).

In yet another preferred embodiment, the composition consists essentially of 5% to 20% by weight glycerin and/or diglycerin, 5% to 20% by weight grapeseed oil, 2% to 30% by weight stearic acid, 0.5% to 5% by weight shea butter, 0.5% to 5% by weight bees wax, 0.5% to 2% by weight cetyl alcohol, 0.1% to 5% by weight preservatives, and balance water and additives.

In still another preferred embodiment, the composition consists essentially of 5% to 20% by weight glycerin and/or diglycerin, 5% to 20% by weight grapeseed oil, 2% to 20% by weight stearic acid, 0.5% to 3% by weight shea butter, 0.5% to 3% by weight bees wax and/or synthetic bees wax, 0.5% to 2% by weight cetyl alcohol, 0.1% to 1% grapefruit seed extract, and the balance water, all weights based on a total weight of the composition.

In another preferred embodiment, the composition consists essentially of 5% to 15% by weight glycerin and/or diglycerin, 5% to 15% by weight grapeseed oil, 2% to 20% by weight stearic acid, 0.5% to 2% by weight shea butter, 0.5% to 2% by Weight bees wax and/or synthetic bees wax, 0.5% to 2% by Weight cetyl alcohol, 0.1% to 3% preservative, 0.5% to 3% orange oil, 0.5% to 3% jojoba oil and the balance water, all weights based on a total weight of the composition.

In yet another preferred embodiment, the composition consists essentially of 5% to 20% by weight glycerin and/or diglycerin, 5% to 20% by weight grapeseed oil, 2% to 20% by weight stearic acid, 0.5% to 2% by weight shea butter, 0.5% to 2% by weight bees wax and/or synthetic bees wax, 0.5% to 2% by weight cetyl alcohol, 0.1% to 3% preservative, 0.5% to 3% soybean oil, 0.5% to 3% jojoba oil and the balance water, all weights based on a total weight of the composition.

In still another preferred embodiment, the composition consists essentially of 2% to 40% by weight glycerin and/or diglycerin, 2% to 40% by weight grapeseed oil and/or soy bean oil, 2% to 40% by weight stearic acid, 0.2% to 10% by weight shea butter, 0.2% to 10% by weight beeswax and/or synthetic beeswax, 0.2% to 10% by weight jojoba oil, 0.20% to 10% by Weight cetyl alcohol, 0.2% to 10% by weight essential oil and/or fragranced oil, 0.1% to 10% by weight preservatives, and the balance water and additives.

All percentage amounts are by weight based on the total weight of the composition, unless stated otherwise.

### EXAMPLES

Amounts listed in the Examples are expressed by weight based on the weight of the total composition. The listed formulas (although intended as indicative) are presented as the presently preferred ingredient content, accurate within plus or minus 5%.

Formulas #1 to #11 were prepared by blending, as described previously (with glyceryl stearate or other appropriate and suitable emulsifier in an amount of 5% or less), the stated ingredients in the stated amounts. One skilled in the art can readily determine the amount of each ingredient in weight (mass) units by deciding upon the desired total weight and calculating the weight of each ingredient from the appropriate percentage listed in each Formula.

### Formula #1; Cream

| | |
|---|---|
| 15% | stearic acid |
| 20% | glycerin or diglycerin |
| 10% | grapeseed oil |
| <5% | glyceryl stearate |
| Balance | water |

### Formula #2: Cream

| | |
|---|---|
| 30% | stearic acid |
| 10% | glycerin or diglycerin |
| <5% | glyceryl stearate |
| Balance | water |

### Formula #3: Cream

| | |
|---|---|
| 25% | stearic acid |
| 15% | grapeseed oil |
| <5% | glyceryl stearate |
| Balance | water |

### Formula #4: Cream

| | |
|---|---|
| 5-10% | grapeseed oil |
| 2-15% | stearic acid |
| <5% | glyceryl stearate |
| 5-20% | glycerin/diglycerin |
| <2% | grapefruit seed extract |
| <2% | Bergamot oil |
| Balance | water |

### Formula #5: Cream

| | |
|---|---|
| 5-20% | glycerin/diglycerin |
| 5-20% | grapeseed oil |
| 2-30% | stearic acid |
| <5% | shea butter |
| <5% | bees wax and/or synthetic bees wax |
| <2% | cetyl alcohol |
| <5% | grapefruit seed extract |
| Balance | water |

### Formula #6: Cream

| | |
|---|---|
| 2-40% | glycerin/diglycerin |
| 2-40% | grapeseed oil |
| 2-40% | soybean oil |
| 2-40% | stearic acid |
| <10% | shea butter |
| <10% | bees wax/synthetic bees wax |
| <10% | jojoba oil |
| <2% | cetyl alcohol |
| <5% | grapefruit seed extract |
| Balance | water |

### Formula #7: Cream

| | |
|---|---|
| 2-40% | glycerin/diglycerin |
| 2-40% | soybean oil |
| 2-40% | stearic acid |
| <10% | shea butter |
| <10% | bees wax/synthetic bees wax |
| <10% | jojoba oil |
| <2% | cetyl alcohol |
| <5% | grapefruit seed extract |
| Balance | water |

### Formula #8: Cream

| | |
|---|---|
| 2-40% | glycerin/diglycerin |
| 2-40% | orange oil |
| 2-40% | stearic acid |
| <10% | shea butter |
| <10% | bees wax/synthetic bees wax |
| <10% | jojoba oil |
| <2% | cetyl alcohol |
| <5% | grapefruit seed extract |
| Balance | water |

### Formula #9: Cream

| | |
|---|---|
| 5-15% | glycerin/diglycerin |
| <2% | carbomer |
| 2-15% | grapeseed oil |
| 2-40% | stearic acid |
| <10% | shea butter |
| <10% | bees wax/synthetic bees wax |
| <10% | jojoba oil |
| <2% | cetyl alcohol |
| <5% | grapefruit seed extract |
| <3% | orange oil |
| <3% | food grade preservative |
| <2% | triethanolamine |
| Balance | water |

### Formula #10: Cream

| | |
|---|---|
| 2-20% | glycerin and/or diglycerin |
| 2-20% | grapeseed oil and/or soybean oil |
| 2-30% | stearic acid |
| <5% | shea butter |
| <5% | bees wax and/or synthetic bees wax |
| <5% | cetyl alcohol |
| <5% | preservatives |
| Balance | water |

### Formula #11: Cream

A mixture of 15 kilograms grapeseed oil, 7.0 kilograms stearic acid, 8.0 kilograms glyceryl stearate SE (SE means self-emulsifying) were heated to 70.degree. C. with good mixing using a propeller mixer to form Phase A. In a separate vessel, about 160 litres of deionized water was heated to 70.degree. C. followed by the addition, with mixing, of 15 kilograms glycerin and 2 litres of grapefruit seed extract to form Phase B. Phase A and Phase B were then mixed together at 70.degree. C. using a propeller mixer and sodium hydroxide was added in sufficient quantity to achieve a pH of 6.25. An emulsion was thereby formed. The resulting emulsion was mixed in a 200 kg capacity Silverson™ high shear mixer for 1 hour then returned to a propeller mixer and allowed to cool to 40.degree. C. The mixer was turned off, the composition was covered and then was allowed to cool to room temperature. Once cooled to room temperature, the cream was transferred to containers for transport.

The cream of Formula #11 had the following composition:

| | |
|---|---|
| 7.5% | grapeseed oil |
| 3.5% | stearic acid |
| 4.0% | glyceryl stearate |
| 7.5% | glycerin |
| 1.0% | grapefruit seed extract |
| Balance | deionized water |

While the present compositions have been described with respect to what is at present considered to be the preferred embodiments, it is to be understood that the invention is not limited to the disclosed embodiments. To the contrary, the invention is intended to cover various modifications, subtractions and/or substitutions included within the spirit and scope of the appended claims.

That which is claimed is:

1. A method for manufacturing a skin care composition comprising:
blending a first phase of the skin care composition, the first phase including a predetermined percentage of water, between about 2% and 20% by weight glycerin and/or diglycerin, and between about 0.1% and 5% by weight of an emulsifier;
heating the first phase to a first predetermined temperature;
blending a second phase of the skin care composition, the second phase including between about 2% to 20% by weight grapeseed oil, between about 2% to 30% by weight stearic acid, between about 0.5% and 5% by weight shea butter, between about 0.5% to 5% by weight beeswax and/or synthetic beeswax, between about 0.5% to 2% by weight cetyl alcohol, and between about 0.5% to 5% by weight essential oil;
heating the second phase to a second predetermined temperature;
adding between about 0.5% to 2.0% of a pH balancer to the first phase of the skin care composition;
blending the first phase at the first predetermined temperature to the second phase at second predetermined temperature; and
cooling the blended first and second phase to a third predetermined temperature;
wherein all weights are based on a total weight of the composition.

2. A method according to claim 1 wherein the first phase and the second phase are combined using shear mixing.

3. A method according to claim 1 wherein the first phase and second phase are blended together until a uniform cream is formed.

4. A method according to claim 1 further comprising stirring the blended first and second phase while cooling to the third predetermined temperature.

5. A method according to claim 1 further comprising blending a predetermined percentage by weight of preservatives and/or additives with the blended first and second phase.

6. A method according to claim 5 wherein the preservatives and additives are blended with the first and second phase using shear mixing.

7. A method according to claim 5 wherein the additives are selected from the group consisting of a perfume, a coloring agent, a UV blocker, a skin protectant, an insect repellent, a medication, a fragranced oil, or any combination thereof.

8. A method according to claim 7 wherein the fragranced oil is orange oil.

9. A method according to claim 1 wherein the emulsifier is one selected from the group consisting of glyceryl stearate and a carbomer; wherein the preservative is grapefruit seed extract and is present in an amount of 0.1% to 2% by weight, and wherein the stearic acid is present in an amount of 10% to 15% by weight.

10. A method according to claim 1 wherein the first predetermined temperature is between about 65 degrees Celsius and 75 degrees Celsius.

11. A method according to claim 1 wherein the second predetermined temperature is between about 70 degrees Celsius and 80 degrees Celsius.

12. A method according to claim 1 wherein the third predetermined temperature is between about 40 degrees Celsius and 50 degrees Celsius.

13. A method for manufacturing a skin care composition comprising:
blending a first phase of the skin care composition, the first phase including a predetermined percentage of water, and between about 2% and 20% by weight glycerin and/or diglycerin;
heating the first phase to a first predetermined temperature;
blending a second phase of the skin care composition, the second phase including between about 2% to 20% by weight grapeseed oil and/or soybean oil, between about 2% to 30% by weight stearic acid, between about 0.5% and 5% by weight shea butter, between about 0.5% to 5% by weight beeswax and/or synthetic beeswax, and between about 0.5% to 2% by weight cetyl alcohol;
heating the second phase to a second predetermined temperature;
blending the first phase at the first predetermined temperature to the second phase at second predetermined temperature; and
cooling the blended first and second phase to a third predetermined temperature;
wherein all weights are based on a total weight of the composition.

14. A method according to claim 13 further comprising blending between about 0.1% and 5% by weight of an emulsifier with the first phase.

15. A method according to claim 13 further comprising adding between about 0.5% to 2.0% by weight of a pH balancer to the first phase of the skin care composition.

16. A method according to claim 13 further comprising adding between about 0.5% to 2.0% by weight of a pH balancer to the blended first and second phase.

17. A method according to claim 13 wherein the first phase and the second phase are combined using shear mixing; wherein the first phase and second phase are blended together until a uniform cream is formed; and further comprising stirring the blended first and second phase while cooling to the third predetermined temperature.

18. A method according to claim 13 further comprising blending a predetermined percentage by weight of preservatives and/or additives with the blended first and second phase; and wherein the preservatives and additives are blended with the first and second phase using shear mixing.

19. A method according to claim 18 wherein the additives are selected from the group consisting of a perfume, a coloring agent, a UV blocker, a skin protectant an insect repellent, a medication, an essential oil, a fragranced oil, or any combination thereof.

20. A method according to claim 13 wherein the emulsifier is one selected from the group consisting of glyceryl stearate and a carbomer, wherein the preservative is grapefruit seed extract and is present in an amount of 0.1% to 2% by weight; and wherein the stearic acid is present in an amount of 10% to 15% by weight.

21. A method according to claim 13 wherein the first predetermined temperature is between about 65 degrees Celsius and 75 degrees Celsius.

22. A method according to claim 13 wherein the second predetermined temperature is between about 70 degrees Celsius and 80 degrees Celsius.

23. A method according to claim 13 wherein the first predetermined temperature is between about 40 degrees Celsius and 50 degrees Celsius.
